(19) United States
(12) Plant Patent Application Publication
(10) Pub. No.: US 2002/0002722 P1
(43) Pub. Date: Jan. 3, 2002

(54) Gladiolus plant named 'Alexandra The Great'

(76) Inventor: John O. Zipperer III, Ft. Myers, FL (US)

Correspondence Address: Laura G. Barrow, Esq., P.O. Box 215, Estero, FL, 33928-0215, US

(21) Appl. No.: 09/877,571

(22) Filed: Jun. 8, 2001

(30) Foreign Application Priority Data
Jun. 9, 2000 (EP) 2000/0888

(51) Int. Cl. A01H 5/00

(52) U.S. Cl. PLT/301

(57) ABSTRACT

A new and distinct gladiolus variety, designated ‘Alexandra The Great’, is shown and described. Compared to the ‘T-200’ variety, the ‘Alexandra The Great’ variety is 30 cm taller and will maintain four to five more flowers in open bloom. The variety is also fable to maintain up to ten flowers in open bloom simultaneously, beginning with a tight cut stem.

The present invention comprises a new and distinct variety of a Gladiolus plant referred to by the variety name ‘Alexandra The Great.’

### BRIEF DESCRIPTION OF THE PHOTOGRAPHS

FIG. 1 is a photograph of a ‘Alexandra The Great’ variety plant in bloom.

FIG. 2 is a photograph of the ‘Alexandra The Great’ variety plant prior to blooming.

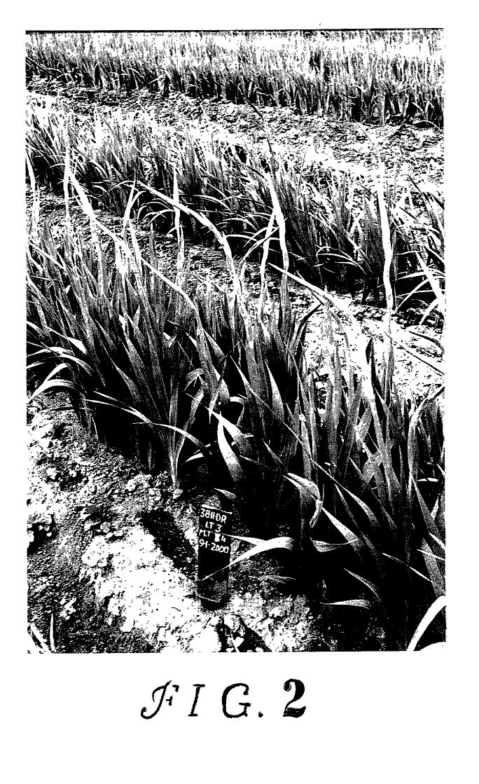

### ASEXUAL REPRODUCTION

The new variety was originated by the Applicant in a controlled proprietary breeding program in Ft. Myers, Fla. wherein selected gladiolus varieties were crossed. (The provisional breeder's designation “38-11” was used in a related application for a Community plant variety right filed earlier in the European Union Community Plant Variety Office). The female parent was a white gladiolus variety named ‘T200,’ an unpatented released variety by Turk characterized in part by having a harvesting time similar to the ‘White Friendship’ variety, 16-18 florets, and lightweight stems. The male parent was a green gladiolus variety named ‘T-815,’ released variety characterized in part by having a long head, small blooms, and poor opening ability. The seeds were planted in Ft. Myers, Fla., and the selection of ‘Alexandra The Great’ variety was made in Spring 1988. Asexual reproduction of the ‘Alexandra The Great’ variety was achieved by collecting cormels from the first corm. All subsequent asexual reproductions of the ‘Alexandra The Great’ variety remain true to the original variety type.

### BOTANICAL DESCRIPTION

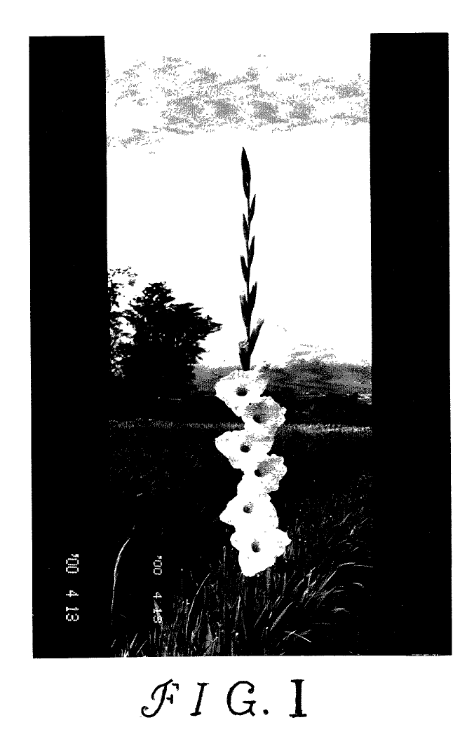

The accompanying color photograph (FIG. 1) shows the inflorescence and various stages of blooming of the ‘Alexandra The Great’ variety plant.

The bud size of the ‘Alexandra The Great’ variety is about 7 cm in length. The flowers of the variety each consist of a total of six petals. Three large petals are oriented on the top half of the bloom and a medium-sized petal is oriented on the lower half of the bloom (at about 6:00). Two smaller petals are oriented on the lower half of the bloom adjacent to the lower medium-sized petal (i.e. at about 4:00 and 8:00). The color of the corolla bloom is a light green deep within the throat of the bloom and a uniform white color extending throughout the rest of the bloom. The diameter of the entire bloom is about 12 cm.

The spike of the plant is about 143 cm and comprises 18-20 florets when grown from #1 size corms (1.5 inch) in Ecuador. The head length is about 60 cm. It should be noted, however, that various factors will affect spike length, including temperature (larger spikes occur in cooler weather), irrigation, light intensity, fertilization, soil type (larger spikes occur in heavy soils versus sandy soil), and bulb size (larger bulbs result in larger spikes). The pistils of the flower are white, and the stamens are brown.

The corms of the ‘Alexandra The Great’ variety are typical for the gladiolus.

The leaf color of the ‘Alexandra The Great’ variety is green. The leaf size is about 67 cm (length) by 4 cm (width).

### GENERAL OBSERVATIONS

The ‘Alexandra The Great’ variety takes about 88 days to harvest in Ecuador, which is about 2-3 days faster than the Advance gladiolus variety. The variety of the present application performs well at all times and tolerates high temperatures well without burning. The variety provides excellent growth in mid-winter days of 10.5 hours.

The variety usually produces one to two stems per Jumbo size corm. The variety can maintain up to ten flowers in open bloom simultaneously in a vase of water and can be harvested with one flower showing color. The variety is an excellent opener after transport, in that the stems may be cut tight, shipped dry for a week, and still bloom well afterwards. The variety is also a fast opener.

The ‘Alexandra The Great’ variety is very resistant to attack by Fusarium fungi, and in fact, is more resistant to Fusarium than the ‘T-200’ parent variety; however, the variety is susceptible to Curvalaria fingi species. The ‘Alexandra The Great’ variety has a medium resistance to foliar diseases (slightly less than the ‘T-200’ variety).

Compared to the T-200 parent variety, the ‘Alexandra The Great’ variety is 30 cm taller, consists of three to four more florets, has a bloom size that is about 1 cm wider in diameter, can hold four to five more blooms open, and is about two weeks slower to harvest. The ‘Alexandra The Great’ variety also has foliage that is a lighter green in color as well as heavier stem.

I claim:

1. A new and distinct gladiolus variety ‘Alexandra The Great’, as shown and described herein.
